SEQUENCE LISTING (9 sequences)

SEQ ID NO: 1 - DNA, 30 bp, Artificial Sequence
  chemically synthesized; designed oligonucleotide primer which is used in the amplification method and hybridizes with pET21a vector DNA
aaccaccatcaaacaggattttcgcctgct

SEQ ID NO: 2 - DNA, 30 bp, Artificial Sequence
  chemically synthesized; designed oligonucleotide primer which is used in the amplification method and hybridizes with pET21a vector DNA
accggatacctgtccgcctttctcccttcg

SEQ ID NO: 3 - DNA, 30 bp, Artificial Sequence
  chemically synthesized;designed oligonucleotide primer which is used in the amplification method and hybridizes with pET21a vector DNA
aaaaccgtctatcagggcgatggcccacta

SEQ ID NO: 4 - DNA, 10 bp, Artificial Sequence
  chemically synthesized; random oligonucleotide primer which is used in the amplification method
ggaaagcgtc

SEQ ID NO: 5 - DNA, 10 bp, Artificial Sequence
  chemically synthesized; random oligonucleotide primer which is used in the amplification method
ggacctcttg

SEQ ID NO: 6 - DNA, 10 bp, Artificial Sequence
  chemically synthesized; random oligonucleotide primer which is used in the amplification method
cacgcagatg

SEQ ID NO: 7 - DNA, 10 bp, Artificial Sequence
  chemically synthesized; random oligonucleotide primer which is used in the amplification method
ttgccttcgg

SEQ ID NO: 8 - DNA, 10 bp, Artificial Sequence
  Dchemically synthesized; random oligonucleotide primer which is used in the amplification method
tcgttcaccc

SEQ ID NO: 9 - DNA, 30 bp, Artificial Sequence
  chemically synthesized; designed oligonucleotide primer which is used in the amplification method and hybridizes with pET21a vector DNA
tagtgggccatcgccctgatagacggtttt

(19) United States
(12) Patent Application Publication
(10) Pub. No.: US 2009/0191595 A1
(43) Pub. Date: Jul. 30, 2009

(54) Method for amplification of long nucleic acid

(75) Inventors: Maiko Tanabe, Tokyo (JP); Hirokazu Nishida, Kokubunji (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 12/071,900

(22) Filed: Feb. 27, 2008

(30) Foreign Application Priority Data
Jun. 20, 2007 (JP) 2007-162929

(51) Int. Cl. C12P 19/34; C07H 21/04

(52) U.S. Cl. 435/91.2; 536/24.33

(57) ABSTRACT

An object of the present invention is to provide a method for amplification of long nucleic acid, wherein the method allows nucleic acid fragments containing the same nucleotide sequence information to efficiently amplify at the same base length. The present invention relates to a method for amplification of long nucleic acid sequence, wherein the method uses primers being modified at the 5′ end with a phosphate group and performs a cooperative reaction using DNA polymerase and DNA ligase.

### CLAIM OF PRIORITY

The present application claims priority from Japanese application JP 2007-162929 filed on Jun. 20, 2007, the content of which is hereby incorporated by reference into this application.

### BACKGROUND OF THE INVENTION

1. Field of the Invention

The present invention relates to a method for amplification of a small amount of sample nucleic acid, the method being useful for genetic analysis and, more specifically, to a method which allows long nucleic acid fragments containing the same nucleotide sequence information to efficiently amplify at the same base length.

2. Background Art

The progress of the molecular biology is described with the advancements in technology dealing with DNA or RNA. In 1985, PCR (Polymerase Chain Reaction) method was developed by Kary Banks Mullis, et al. (R. K. Saiki, et al., Science, Vol. 239, pp. 487-491 (1988)) and introduced in the fields of molecular biology research. PCR method enabled researchers to amplify a specific region of a DNA template exponentially. The method brought a revolution to many research fields ranging from virus identification to transcriptional regulation.

The conventional PCR method is insufficient these days, and an analysis on a genome-wide scale is required to take over. For example, in the field of genetic typing, which is routinely used in the field of pathology research, there are difficulties in obtaining a sufficient amount of genetic resources. This is becoming an issue in genetic typing. The preservation of genetic material is not confined to genotyping, but is also putting a restriction on research achievements in many fields such as drug discovery and functional genomics. Even though the conventional PCR method can amplify a target sequence fragment, the resulting fragments do not adequately represent a whole genome.

Researchers have developed innovative technologies which enabled them to conduct whole genome amplification to solve the above problem since the early 1990s. Examples of whole genome amplification methods which were developed in the initial stages include PEP (Primer Extension Preamplification) method (KangPu Xu, et al., Human Reproduction, Vol. 8, pp. 2206-2210 (1993)), DOP (Degenerate Oligonucleotide-Primed) PCR method (Hakan Telenius, et al., Genomics, Vol. 13, pp. 718-725 (1992)), and TPCR (Tagged PCR) method (Dietmar Grothues, et al., Nucl. Acids Res. Vol. 21, pp. 1321-1322 (1993)). Any of the above has proven to be useful as an approach, but had a problem in terms of relatively short amplified products (approximately 500-nucleotide length) and redundancy in nucleotide sequence information of the resulting amplified products in comparison with that of the template.

Subsequently, several other methods such as Genome Plex WGA (Whole Genome Amplification) method (U.S. Patent No. 2003/0143599) and MDA (Multiple Displacement Amplification) method (David L. Barker, et al., Genome Res. Vol. 14, pp. 901-907 (2004)) were developed. Compared to the early stage, these methods showed the increased base length of amplified products and had a reduced redundancy in nucleotide sequence information reproduced by the resulting amplified products. However, with the methods, the same sequence information is shared by a plurality of amplified products of different lengths. Therefore, even when the amplified products resulting from the above whole genome amplification method are used, it is concerned that the difference in redundancy of nucleotide sequence information stored by the amplified products may affect the analysis results. Therefore, development of technique of whole genome amplification has been awaited in order to overcome such drawbacks.

### SUMMARY OF THE INVENTION

A primary object of the present invention is to provide a method that allows amplification wherein the length of nucleic acid fragments containing the same nucleotide sequence information is always kept constant, which overcomes the drawbacks of conventional whole genome amplification techniques.

The present inventors have developed a method for amplification of nucleic acid for a region of unknown sequence, and minimize the redundancy of the amplified products. This method allows amplification of long nucleic acid fragments containing the same nucleotide sequence information in the nucleic acid fragments of the same length for an unknown sequence region by using the plurality of random primers, which modified phosphate group at the 5′ ends and by cooperatively reacting DNA polymerase and DNA ligase.

More specifically, the present invention relates to a method for amplification of nucleic acid containing a first step of hybridizing a plurality of primers having a phosphate group at the 5′ ends, a second step of elongating the primers hybridized to the sample nucleic acid using DNA polymerase, a third step of ligating adjacent elongation products by DNA ligase to generate replicated strand complementary to the sample nucleic acid, and a fourth step of dissociating the replicated strand from the sample nucleic acid.

In the second embodiment, the first to fourth steps performed in succession in this order, followed by the first step.

In the third embodiment, the fifth step performed to dissociate the elongation products and the sample nucleic acid after the first step and the second step. Subsequently, the first, second and fifth steps are repeated using the sample nucleic acid dissociated from the elongation products in the fifth step, followed by the first to third steps.

In the method according to the present invention, plurality of primers is random primers, which are not designed specifically to the sample nucleic acid.

According to the present invention, preferably DNA polymerase preferably does not possess strand displacement ability. Also, preferably DNA ligase which is which does not possess blunt-end ligation ability and is preferably heat-resistant.

Furthermore, the present invention also provides a primer set consisting of a plurality of random primers, which modified phosphate group at the 5′ ends. In addition, the present invention also provides a kit of nucleic acid amplification, which is contained a plurality of random 5′ end phosphate group modified primers, DNA polymerase which does not possess strand displacement ability, and a heat-resistant DNA ligase which does not possess blunt-end ligation ability.

According to the present invention, the amplification of long nucleic acid fragments containing the identical nucleotide sequence information, and it is made possible to perform an analysis similar to the analysis conducted with the conventional genome samples.

### BRIEF DESCRIPTION OF THE DRAWINGS

FIG. 1 shows the procedure according to the first embodiment of the present invention;

FIG. 2 shows the procedure according to the second embodiment of the present invention;

FIG. 3 shows the procedure according to the third embodiment of the present invention.

FIG. 4 shows the procedure of amplification according to the method of the present invention;

FIG. 5 shows the results of analyzing the amplified products by electrophoresis, the amplified products being obtained by the procedure of the first and second embodiments of the present invention using pET21a vector DNA as a template;

FIG. 6 shows the results of analyzing the amplified products by electrophoresis, the amplified products being obtained by the procedure of the first and second embodiments of the present invention using pET21a vector DNA as a template and random primers;

FIG. 7 shows the procedure of amplification according to the method of the present invention;

FIG. 8 shows the results of analyzing the amplified products by electrophoresis, which being obtained by the procedure of the third embodiment of the present invention using pET21a vector DNA as a template;

FIG. 9 shows the results of analyzing the amplified products by electrophoresis, the amplified products being obtained by the procedure of the third embodiment of the present invention using pET21a vector DNA as a template and random primers;

FIG. 10 shows the procedure of amplification according to the method of the present invention; and

FIG. 11 shows the results of analyzing the PCR products by electrophoresis, which being obtained using pET21a vector DNA or the reaction products of Example 2 as a template.

### DETAILED DESCRIPTION OF THE PREFERRED EMBODIMENTS

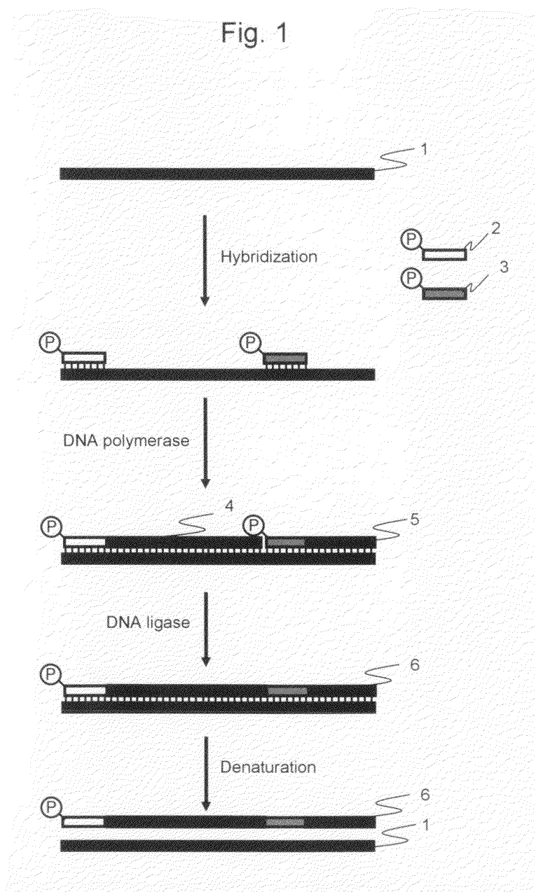

FIG. 1 shows the procedure according to the first embodiment of the present invention. The present invention relates to a method for amplification of long nucleic acid fragments (for example, 10000-nucleotide length or longer, and preferably 100000-nucleotide length or longer, which are difficult to amplify by the conventional PCR method). The method of the present invention possesses a first step of hybridizing Primers 2 and 3 which comprises a sequence complementary to the single-strand Sample nucleic acid 1 and modified phosphate group at the 5′ ends, a second step of elongating the primers hybridized in the above first step using DNA polymerase, a third step of ligating adjacent Elongation Products 4 and 5 being obtained in the second step by DNA ligase, and a fourth step of dissociating Replicated Strand 6 being hybridized to single-strand Sample nucleic acid 1 by heat denaturation.

Through ligation of elongation products derived from the primers by DNA ligase, it is made possible to induce no variation in length of nucleic acid fragments containing the same nucleotide sequence information as well as to obtain nucleic acid fragments being longer than the nucleotide length of elongation limit of DNA polymerase.

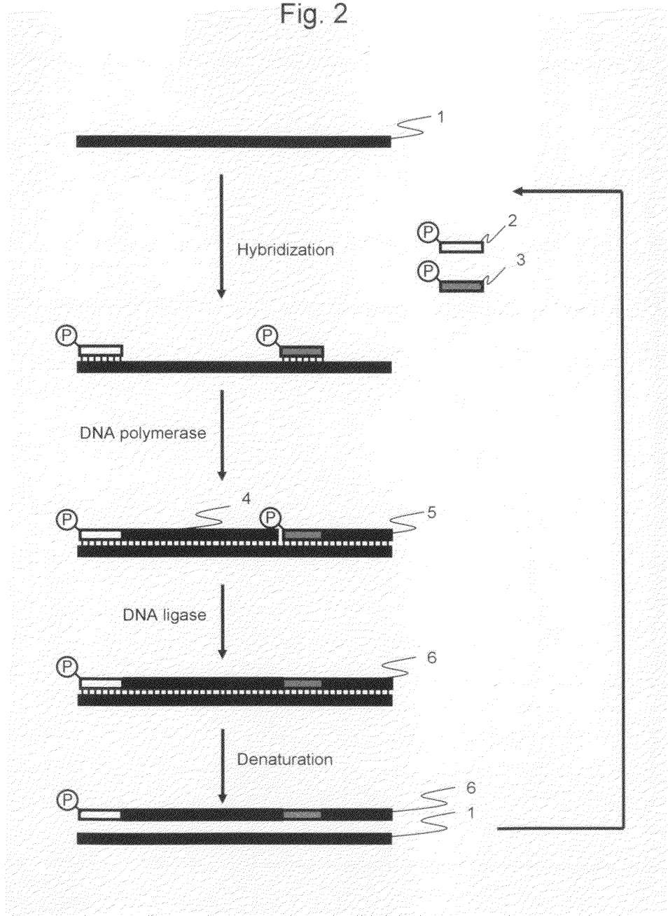

FIG. 2 shows the procedure according to the second embodiment of the present invention. The present invention relates to a method for amplification of long nucleic acid fragments. The method of the present invention possesses a first step of hybridizing Primers 2 and 3 which comprises a sequence complementary to the single-strand Sample nucleic acid 1 and modified phosphate group at the 5′ ends, a second step of elongating the primers hybridized in the above first step using DNA polymerase, a third step of ligating adjacent Elongation Products 4 and 5 being obtained in the second step by DNA ligase, a fourth step of dissociating Replicated Strand 6 being hybridized to single-strand Sample nucleic acid 1 by heat denaturation, and the first, second, third, and fourth steps are repeated in succession using single-strand Sample nucleic acid 1 being obtained in the fourth step as a template.

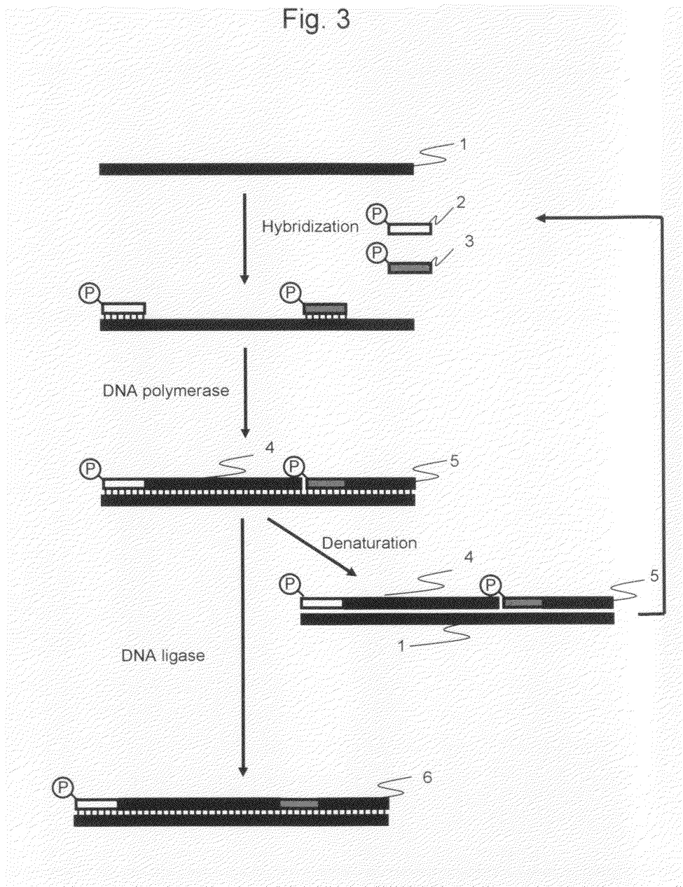

FIG. 3 shows the procedure according to the third embodiment of the present invention. The present invention relates to a method for amplification of long nucleic acid fragments and gene amplification method, the method repeating a first step of hybridizing Primers 2 and 3 which possess single-strand Sample nucleic acid 1 and modified phosphate group at the 5′ ends, a second step of elongating the primers hybridized in the above first step using DNA polymerase, and a fifth step of dissociating adjacent Elongation Products 4 and 5 being obtained in the second step from single-strand Sample nucleic acid 1 in succession, followed by a third step of ligating adjacent Elongation Products 4 and 5 by DNA ligase.

By employing a method wherein each step of the second and third embodiments of the present invention is repeated in succession, a sample nucleic acid in a concentration approximately equivalent to the amount of a template which can be amplified by the conventional PCR method can be amplified by more than a few hundred-fold. This makes it possible to replicate a trace amount of sample nucleic acid. Furthermore, according to the procedures of the third embodiment, in the case where the final products are required to be a single-strand sample nucleic acid, a step of dissociating Replicated Strand 6 being hybridized to single-strand Sample nucleic acid 1 by heat denaturation can be added.

According to the procedures of the first, second, and third embodiments of the present invention, the number of primers which are used in the first step is plural greater than or equal to two, and can include either forward or reverse primers. Furthermore, in the case where the template sequence cannot be identified, multiple kinds of random primers can be employed to perform the reaction to make it possible to amplify long nucleic acid fragments having unknown sequences without preparing primers having a template-specific sequence.

The term “random primer” used herein refers to a synthetic primer generally consisting of 6 to 30 mer nucleic acid and being generated by randomly assembling Adenine (A), Thymine (T), Guanine (G) and Cytosine (C), and is not designed to a specific sequence in the sample nucleic acid to be amplified. Any of the random primers hybridizes with the complimental site in the template in order to serve as a primer for the replication of the template when it reacts in accordance with the flows of the first, second and third embodiments. Moreover, random primers can be selected from conventionally used primers. Examples of conventional random primers include 10 mer random primers which are commercially available from Operon Technologies, Inc. and DNA oligomer set (12 mer) commercially available from Wako Pure Chemical Industries, Ltd.

According to the procedures of the first, second, and third embodiments of the present invention, DNA polymerase which is used in the second step of the present invention preferably has heat resistance and no strand displacement ability. Examples of such DNA polymerase include Pfu DNA polymerase, Taq DNA polymerase, E. coli DNA polymerase I, ΔTth DNA polymerase, T7 DNA polymerase, T4 DNA polymerase, Kod DNA polymerase, and Pyrobest (registered trademark) DNA polymerase.

According to the procedures of the first, second, and third embodiments of the present invention, DNA ligase which is used in the third step of the present invention preferably has heat resistance and no blunt end ligation activity. This spec prevents the occurrence of nonspecific binding between nucleic acid fragments. Also, regarding the necessity of having heat resistance, the reaction of the present invention is required to be conducted at a relatively high temperature (60 to 90° C.) in order to amplify relatively long nucleic acid lengths (for example, 10000-nucleotide length or longer, and preferably 100000-nucleotide length or longer). Examples of such DNA ligase include Pfu DNA ligase, Tth DNA polymerase, Rma DNA ligase, Tsc DNA ligase, and E. coli DNA ligase.

In the present invention, either a thermal cycle reaction or an isothermal reaction can be employed for the procedures of the first, second, and third embodiments. Also, according to the procedures of the first, second, and third embodiments of the present invention, the nucleotide sequence which is used as a template may be an RNA sequence. In the case where the sequence used as a template is DNA, either single-strand DNA or double-strand DNA can be used. When double-strand DNA is used as a template, the method of the present invention should be carried out following a process of pretreatment step, so as to denature the double-strand DNA into single strand DNA.

### EXAMPLES

The present invention is hereafter described in greater detail with reference to the examples, despite the technical scope of the present invention is not limited to these examples.

### Example 1

### 1. Oligonucleotide Primers Used in Example 1

Primer 1:

(SEQ ID NO: 1)

5′-AACCACCATCAAACAGGATTTTCGCCTGCT-3′
Primer 2:

(SEQ ID NO: 2)

5′-ACCGGATACCTGTCCGCCTTTCTCCCTTCG-3′
Primer 3:

(SEQ ID NO: 3)

5′-AAAACCGTCTATCAGGGCGATGGCCCACTA-3′

The amplified products obtained by the cooperative reaction, which were performed the elongation reaction and ligation reaction using DNA polymerase and DNA ligase cooperatively (hereinafter referred to as “cooperative reaction”) during isothermal reaction, were analyzed by electrophoresis in order to determine whether or not the long nucleic acid fragments amplification could be carried out in accordance with the procedure according to the first and second embodiments of the present invention.

pET21a vector DNA (Takara Bio Inc.) treated with a restriction enzyme HpaI (concentration: 25 ng/μL) was used as a template, and the primers described in the above 1 were used as the oligonucleotide primers for amplification. The HpaI cleavage site of pET21a vector DNA be defined as the first nucleotide, Primer 1 was a forward primer having a sequence which was complementary to a region between nucleotide positions 1 to 30. Primer 2 was a forward primer having a sequence which was complementary to a region between nucleotide positions 1801 to 1830. Primer 3 was a forward primer having a sequence which was complementary to a region between nucleotide positions 3601 to 3630. Primers 1, 2, and 3 were all labeled at the 5′ end with a phosphate group.

In relation to the composition for the amplification reaction, Pfu Ultra High-Fidelity DNA polymerase (Stratagene, Inc.) was used as DNA polymerase, and Pfu DNA ligase (Stratagene, Inc.) and the accompanying buffer were used as DNA ligase and a reaction buffer. The amounts of the enzymes were determined in accordance with the instruction manual for each enzyme. The amounts of dNTPs and primers were determined in accordance with the instruction manual for Pfu DNA polymerase.

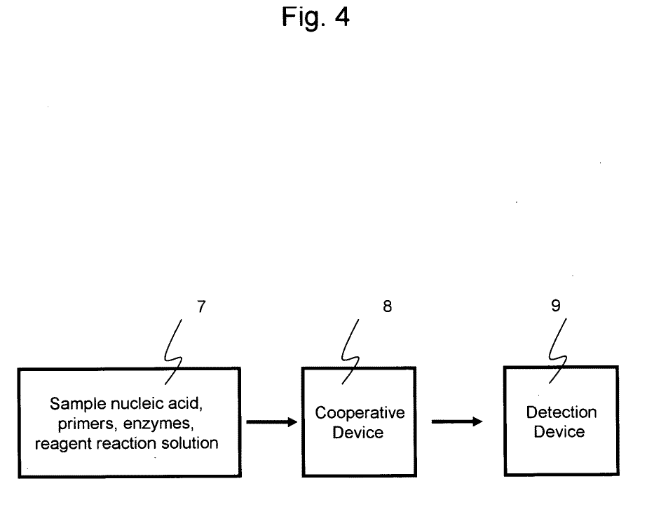

FIG. 4 shows a procedure of amplification according to the method of the present invention. Reaction Solution 7 comprising a sample nucleic acid, primers, enzymes, reagents and the like. Reaction Solution 7 was denatured at 95° C. for 5 minutes, followed by 24 cycles of treatment, each cycle consisting of 95° C. for 30 seconds, 37° C. for 30 seconds, 70° C. for 4 minutes, 95° C. for 30 seconds and 70° C. for 10 minutes. The resulting reaction products were subjected to electrophoresis in 1.5% agarose gel. These reactions were carried out in Cooperative Reaction Device 8, and the reaction products were detected in Detection Device 9 by staining the agarose gel with SYBR Gold (Molecular Probes, Inc.). GeneAmp PCR System 9700 (Applied Biosystems, Inc.) was used as Cooperative Reaction Device 8, and FluorImager 595 (GE Healthcare, Ltd.) was used as Detection Device 9.

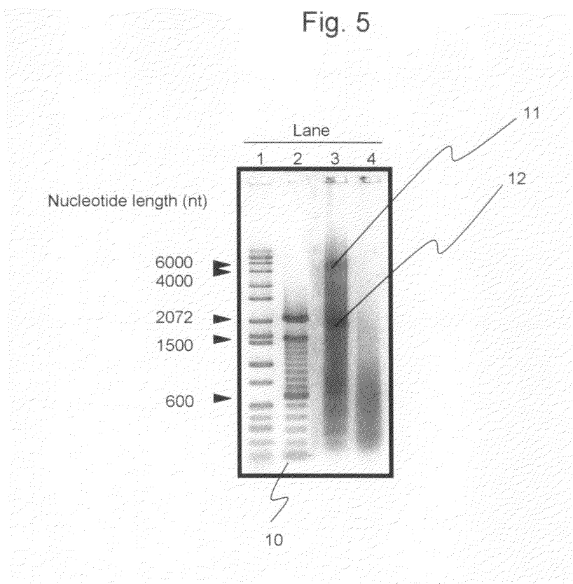

Electrophoresis Image 10 in FIG. 5 shows the results of an electrophoresis in accordance with the procedure according to the first and second embodiments of the present invention. Lane 1 is the Hi-Lo DNA marker (Abetech, Inc.), Lane 2 is a 100 bp ladder (Invitrogen, Inc.), Lane 3 is the above reaction product, and Lane 4 is a negative control (without template). As a result, it was confirmed that Amplified Band 11 appeared at the position similar to the template consisting of 5443 nucleic acids in length in Lane 3, but the amplified band did not appear in Lane 4. Moreover, it was confirmed that Amplified Band 12, which corresponded to the unbound elongation product consisting of approximately 1800 nucleic acids in length, appeared in Lane 3. This indicates that the amplified products of interest can be obtained by the procedure according to the first and second embodiments of the present invention. Further, such results confirmed that according to the present invention, it is made possible to amplify long nucleic acid fragments.

### Example 2

### 1. Oligonucleotide Primers Used in Example 2

| | |
|---|---|
| Primer 4: (AE07) | |
| 5′-GGAAAGCGTC-3′ | (SEQ ID NO: 4) |
| Primer 5: (AA12) | |
| 5′-GGACCTCTTG-3′ | (SEQ ID NO: 5) |
| Primer 6: (AZ17) | |
| 5′-CACGCAGATG-3′ | (SEQ ID NO: 6) |
| Primer 7: (AA20) | |
| 5′-TTGCCTTCGG-3′ | (SEQ ID NO: 7) |
| Primer 8: (AE02) | |
| 5′-TCGTTCACCC-3′ | (SEQ ID NO: 8) |

The reaction products were analyzed by electrophoresis in order to determine that whether or not the same amplified products could be obtained using random primers as the primers in accordance with the procedure according to the first and second embodiments of the present invention.

pET21a vector DNA (Takara Bio Inc.) treated with a restriction enzyme HpaI (concentration: 25 ng/μL) was used as a template, and the primers described in the above 1 were used as the oligonucleotide primers for amplification. Five kinds of primers (AA12, AA20, AE02, AE07, and AZ17) commercially available from Operon Technologies, Inc. were modified with T4 Polynucleotide Kinase (Takara Bio Inc.) to add a phosphate group at the 5′ end. The protocol for the modification reaction to add a phosphate group was determined in accordance with the instruction manual for the enzyme.

In relation to the composition for the amplification reaction, Pfu DNA polymerase (Stratagene, Inc.) was used as DNA polymerase, and Pfu DNA ligase (Stratagene, Inc.) and the accompanying buffer were used as DNA ligase and a reaction buffer. The amounts of the enzymes used were determined in accordance with the instruction manual for each enzyme. The amounts of dNTPs and primers were determined in accordance with the instruction manual for Pfu DNA polymerase.

FIG. 4 shows a procedure of amplification according to the method of the present invention. Reaction Solution 7 comprising a sample nucleic acid, primers, enzymes, reagents and the like. The Reaction Solution 7 was denatured at 95° C. for 5 minutes in Cooperative Reaction Device 8, followed by 24 cycles of treatment, each cycle consisting of 95° C. for 30 seconds, 37° C. for 30 seconds, 70° C. for 2 minutes, 95° C. for 30 seconds and 70° C. for 10 minutes, and resulting reaction products were subjected to electrophoresis in 1.5% agarose gel. These reactions were carried out in Cooperative Reaction Device 8, and the reaction products were detected in Detection Device 9 by staining the agarose gel with SYBR Gold (Molecular Probes, Inc.). GeneAmp PCR System 9700 (Applied Biosystems, Inc.) was used as Cooperative Reaction Device 8, and FluorImager 595 (GE Healthcare, Ltd.) was used as Detection Device 9.

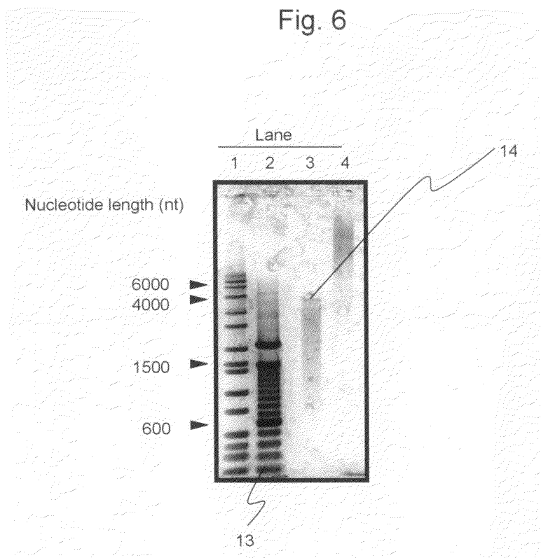

Electrophoresis Image 13 in FIG. 6 shows the results of an electrophoresis in Example 2. Lane 1 is the Hi-Lo DNA marker (Abetech, Inc.), Lane 2 is a 100 bp ladder (Invitrogen, Inc.), Lane 3 is the above reaction product, and Lane 4 is a negative control (without template). As a result, it was confirmed that Amplified Band 14 appeared at the position of 5443-nucleotide length, which was equivalent to the nucleotide length of the template, in Lane 3, but the amplified band did not appear in Lane 4. These results suggest that, with the methods of the first and second embodiments of the present invention, the amplified products of interest can also be obtained using random primers. Further, such results confirmed that according to the present invention, it is made possible to amplify long nucleic acid fragments for a nonspecific region.

### Example 3

### 1. Oligonucleotide Primers Used in Example 3

Primer 1:

(SEQ ID NO: 1)

5′-AACCACCATCAAACAGGATTTTCGCCTGCT-3′
Primer 2:

(SEQ ID NO: 2)

5′-ACCGGATACCTGTCCGCCTTTCTCCCTTCG-3′
Primer 3:

(SEQ ID NO: 3)

5′-AAAACCGTCTATCAGGGCGATGGCCCACTA-3′

The amplified products were analyzed by electrophoresis in order to determine whether or not long nucleic acid fragments amplification could be carried out in accordance with the procedure according to the third embodiment of the present invention.

pET21a vector DNA (Takara Bio Inc.) treated with a restriction enzyme HpaI (concentration: 25 ng/μL) was used as a template, and the primers described in the above 1 were used as the oligonucleotide primers for amplification. The HpaI cleavage site of pET21a vector DNA be defined as the first nucleotide, Primer 1 was a forward primer having a sequence which was complementary to a region between nucleotide positions 1 to 30. Primer 2 was a forward primer having a sequence which was complementary to a region between nucleotide positions 1801 to 1830. Primer 3 was a forward primer having a sequence which is complementary to a region between nucleotide positions 3601 to 3630. Primers 1, 2, and 3 were all labeled phosphate group at the 5′ end. In relation to the composition for the amplification reaction, Pfu DNA polymerase (Stratagene, Inc.) was used as DNA polymerase, and Pfu DNA ligase (Stratagene, Inc.) and the accompanying buffer were used as DNA ligase and a reaction buffer. The amounts of the enzymes used were determined in accordance with the instruction manual for each enzyme. The amounts of dNTPs and primers were determined in accordance with the instruction manual for Pfu DNA polymerase.

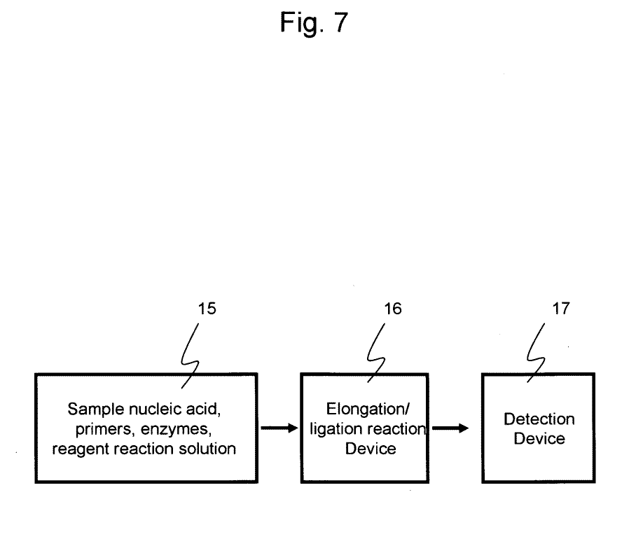

FIG. 7 shows a procedure of amplification according to the method of the present invention. Reaction Solution 15 comprising a sample nucleic acid, primers, enzymes, reagents and the like. The Reaction Solution 15 was denatured at 95° C. for 5 minutes in Elongation/Ligation Reaction Device 16, followed by 30 cycles of treatment, each cycle consisting of 95° C. for 30 seconds, 60° C. for 30 seconds and 72° C. for 4 minutes to conduct an elongation reaction. Subsequently, the elongation products were treated at 70° C. for 4 hours to perform a ligation reaction thereof. The resulting reaction products were subjected to electrophoresis in 1.5% agarose gel. The reaction products were detected in Detection Device 17 by staining the agarose gel with SYBR Gold (Molecular Probes, Inc.), and GeneAmp PCR System 9700 (Applied Biosystems, Inc.) was used as Elongation/Ligation Reaction Device 16, and FluorImager 595 (GE Healthcare, Ltd.) was used as Detection Device 17.

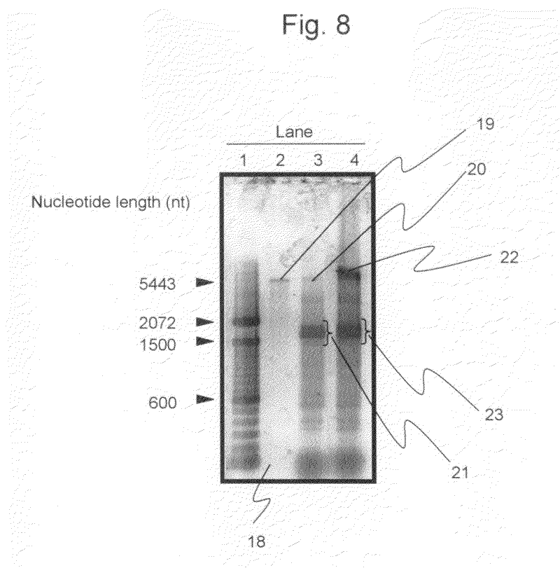

Electrophoresis Image 18 in FIG. 8 shows the results of an electrophoresis in Example 3. Lane 1 shows a 100 bp ladder (Invitrogen, Inc.), Lane 2 contains only the template, Lane 3 shows the reaction product which was not subjected to the ligation reaction at 70° C. for 4 hours, and Lane 4 shows the reaction product which was subjected to the ligation reaction. As a result, it was confirmed that Amplified Band 20 in Lane 3 and Amplified Band 22 in Lane 4, both of which were at the same position as Amplified Band 19 of the template consisting of 5443 nucleic acids in length. By comparison between Amplified Band 20 and Amplified Band 22, there was obviously an increase in the level of amplified products in Amplified Band 22. Moreover, there were two amplified bands, Amplified Band 21 and Amplified Band 23, which are composed of the unbound elongation products consisting of 1800 nucleic acids and 1843 nucleic acids in length, in both Lane 3 and Lane 4. These results suggest that the amplified product of interest can be obtained by procedure according to the third embodiment of the present invention. And such results confirmed that according to the present invention, it is made possible to amplify long nucleic acid fragments.

### Example 4

### 1. Oligonucleotide Primers Used in Example 4

| | |
|---|---|
| Primer 4: (AE07) | |
| 5′-GGAAAGCGTC-3′ | (SEQ ID NO: 4) |
| Primer 5: (AA12) | |
| 5′-GGACCTCTTG-3′ | (SEQ ID NO: 5) |
| Primer 6: (AZ17) | |
| 5′-CACGCAGATG-3′ | (SEQ ID NO: 6) |
| Primer 7: (AA20) | |
| 5′-TTGCCTTCGG-3′ | (SEQ ID NO: 7) |
| Primer 8: (AE02) | |
| 5′-TCGTTCACCC-3′ | (SEQ ID NO: 8) |

The amplified products were analyzed by electrophoresis in order to determine whether or not the same amplified products of interest can be obtained using random primers as the primers in accordance with the procedure according to third embodiment of the present invention.

pET21a vector DNA (Takara Bio Inc.) treated with a restriction enzyme HpaI (concentration: 25 ng/μL) was used as a template, and the primers described in the above 1 were used as the oligonucleotide primers for amplification. Five kinds of primers (AA12, AA20, AE02, AE07, and AZ 17) commercially available from Operon Technologies, Inc. were modified with T4 Polynucleotide Kinase (Takara Bio Inc.) to add a phosphate group to the 5′ end to be used as random primers. The protocol for the modification reaction to add a phosphate group was determined in accordance with the instruction manual for the enzyme.

In relation to the composition for the amplification reaction, Pfu DNA polymerase (Stratagene, Inc.) was used as DNA polymerase, and Pfu DNA ligase (Stratagene, Inc.) was used as DNA ligase. As for the buffer, Pfu clone buffer (200 mM Tris-HCl (pH 8.8), 20 mM MgSO₄, 100 mM KCl, 100 mM (NH₄)₂SO₄, 1% Triton X-100, 1 mg/mL nuclease-free BSA, 5 mmol ATP) was used. The amounts of the enzymes used were determined in accordance with the instruction manual for each enzyme. The amounts of dNTPs and primers were determined in accordance with the instruction manual for Pfu DNA polymerase.

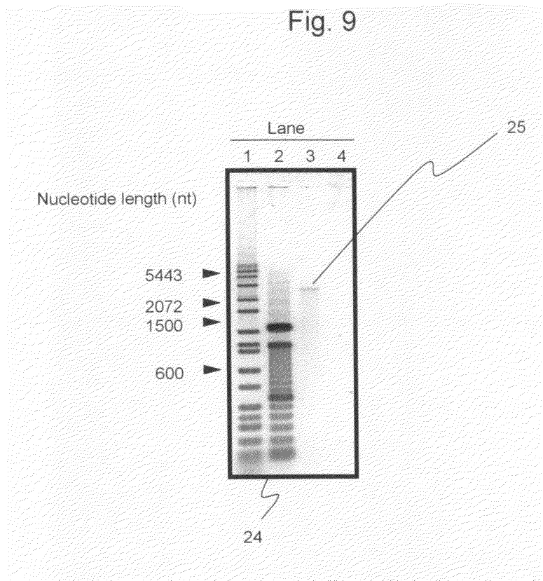

FIG. 7 shows a procedure of amplification according to the method of the present invention. Reaction Solution 15 comprising a sample nucleic acid, primers, enzymes, reagents and the like was denatured at 95° C. for 5 minutes in Elongation/Ligation Reaction Device 16, followed by 30 cycles with each cycle consisting of 95° C. for 30 seconds, 37° C. for 30 seconds and 72° C. for 2 minutes to conduct an elongation reaction. Subsequently, the elongation products were treated at 70° C. for 4 hours to perform a ligation reaction thereof. The resulting reaction products were subjected to electrophoresis in 1.5% agarose gel. Subsequently, the reaction products were detected in Detection Device 17 by staining the agarose gel with SYBR Gold (Molecular Probes, Inc.). GeneAmp PCR System 9700 (Applied Biosystems, Inc.) was used as Elongation/Ligation Reaction Device 16, and Fluorlmager 595 (GE Healthcare, Ltd.) was used as Detection Device 17. Electrophoresis Image 24 in FIG. 9 shows the results of an electrophoresis in Example 4. Lane 1 is the Hi-Lo DNA marker (Abetech, Inc.), Lane 2 is a 100 bp ladder (Invitrogen, Inc.), Lane 3 is the above reaction product, and Lane 4 is a negative control which was subjected to a similar reaction without the addition of the template. As a result, it was confirmed that Amplified Band 25 appeared at the position of 5443-nucleotide length, which was equivalent to the nucleotide length of the template, in Lane 3, but the amplified band did not appear in Lane 4. These results suggest that the amplified products of interest can also be obtained using random primers with the method of the third embodiment of the present invention. And such results confirmed that according to the present invention, it is made possible to amplify long nucleic acid fragments for a nonspecific region.

### Example 5

### 1. Oligonucleotide Primers Used in Example 5

Primer 2:

(SEQ ID NO: 2)

5′-ACCGGATACCTGTCCGCCTTTCTCCCTTCG-3′
Primer 9:

(SEQ ID NO: 9)

5′-TAGTGGGCCATCGCCCTGATAGACGGTTTT-3′

### 2. PCR Reaction Composition Used in Example 5

KOD Dash buffer (×1), template (5 μl of the reaction product obtained in Example 2, 10 ng of pET21a/HpaI), primers (10 pmol each), dNTPs (0.2 mM), enzyme (1.88 U)

The amplified products were analyzed by electrophoresis in order to determine whether or not the same amplified products of interest can be obtained using the two products as a template, the reaction product obtained in Example 2 and pET21a/HpaI, for the PCR method in accordance with the procedure according to first and second embodiments of the present invention.

The amplified products obtained in Example 2 and pET21a vector DNA (Takara Bio Inc.) treated with a restriction enzyme HpaI (concentration: 10 ng/μL) were used as templates, and the primers described in the above 1 were used as the oligonucleotide primers for amplification. The HpaI cleavage site of pET21a vector DNA be defined as the first nucleotide, Primer 2 was a forward primer having a sequence which was complementary to a region between nucleotide positions 1801 to 1830. Primer 9 was a forward primer having a sequence which was complementary to a region between nucleotide positions 3601 to 3630. Primers 2 and 9 were both labeled at the 5′ end with a phosphate group.

In relation to the PCR composition, the composition described in the above 2 was used. KOD Dash DNA polymerase (Toyobo Co., Ltd.) was used as DNA polymerase.

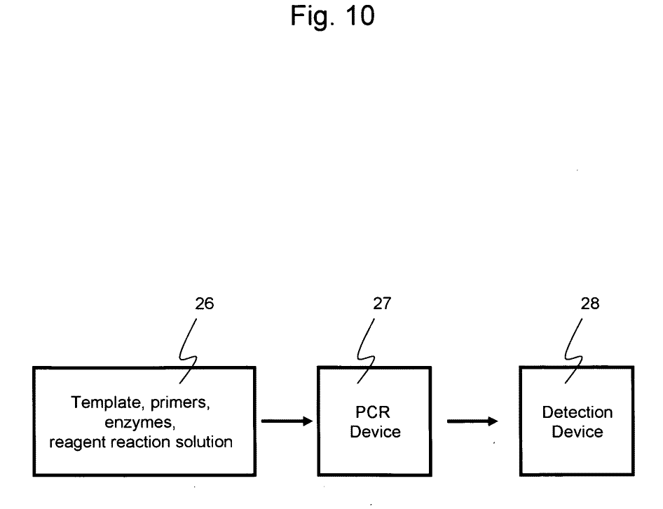

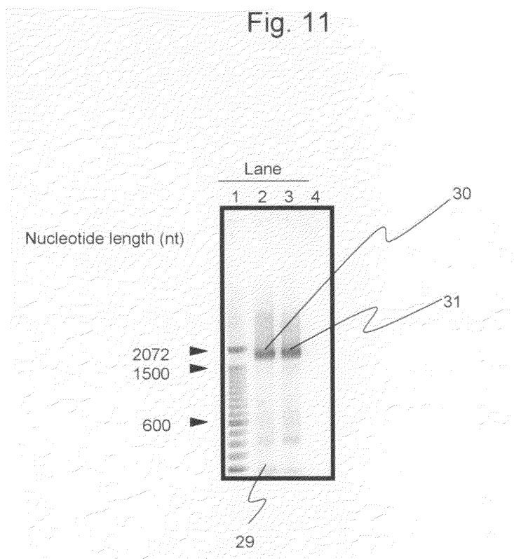

FIG. 10 shows a procedure of amplification according to the method of the present invention. Reaction Solution 26 comprising template, random primers, enzymes, reagents and the like was denatured at 94° C. for 4 minutes in PCR Device 27, followed by 24 cycles of treatment, each cycle consisting of 94° C. for 30 seconds, 60° C. for 2 seconds and 74° C. for 30 seconds. The resulting reaction products were subjected to electrophoresis in 1.5% agarose gel. Subsequently, the reaction products were detected in Detection Device 28 by staining the agarose gel with SYBR Gold (Molecular Probes, Inc.). GeneAmp PCR System 9700 (Applied Biosystems, Inc.) was used as PCR Device 27, and FluorImager 595 (GE Healthcare, Ltd.) was used as Detection Device 28. Electrophoresis Image 29 in FIG. 11 shows the results of an electrophoresis in Example 5. Lane 1 is a 100 bp ladder (Invitrogen, Inc.), Lane 2 is the reaction product obtained using pET21a vector DNA (Takara Bio Inc.) treated with a restriction enzyme HpaI as a template, Lane 3 is the same amplified product obtained using the reaction product in Example 2 as a template, and Lane 4 is a negative control which was subjected to a similar reaction without the addition of the template. As a result, it was confirmed that Amplified Bands 30 and 31 appeared at the position of 1800-nucleotide length, which corresponded to the position of the amplified product of interest, in Lane 2 and Lane 3. These results suggest that the amplified products of interest can also be obtained using a conventional sample nucleic acid with the method of the present invention. And such results confirmed that according to the present invention, it is made possible to amplify a sample nucleic acid.

According to the present invention, it is made possible to amplify long nucleic acid fragments containing the identical nucleotide sequence information for a nonspecific region. Therefore, the present invention is useful in the fields of life science and the like, wherein whole genome analysis is required.

What is claimed is:

1. A method for amplification of nucleic acid comprising a first step of hybridizing a plurality of primers having a phosphate group at the 5′ end to a sample nucleic acid, a second step of elongating the primers hybridized to the sample nucleic acid using DNA polymerase, a third step of ligating adjacent elongation products by DNA ligase to generate replicated strand complementary to the sample nucleic acid, and a fourth step of dissociating the replicated strand from the sample nucleic acid.

2. The method for amplification of nucleic acid according to claim 1, wherein the first to fourth steps are performed in succession in this order, followed by repeating the first to fourth steps using the sample nucleic acid dissociated from the replicated strand in the fourth step.

3. The method for amplification of nucleic acid according to claim 1, wherein the fifth step is performed to dissociate the elongation products and the sample nucleic acid after the first step and the second step, and subsequently, the first, second and fifth steps are repeated using the sample nucleic acid dissociated from the elongation products in the fifth step, followed by performing the first to third steps.

4. The method for amplification of nucleic acid according to claim 1, wherein the plurality of primers are random primers, the primers being not designed specifically to the sample nucleic acid.

5. The method for amplification of nucleic acid according to claim 1, wherein the DNA polymerase does not possess strand displacement ability.

6. The method for amplification of nucleic acid according to claim 1, wherein the DNA ligase does not possess blunt-end ligation ability.

7. The method for amplification of nucleic acid according to claim 1, wherein the DNA ligase is a heat-resistant DNA ligase.

8. A primer set comprising a plurality of random primers having a phosphate group at the 5′ end.

9. A kit of nucleic acid amplification comprising a plurality of random primers having a phosphate group at the 5′ end, DNA polymerase without strand displacement ability, and a heat-resistant DNA ligase without blunt-end ligation ability.
